(19) United States
(12) Patent Application Publication
(10) Pub. No.: US 2019/0255130 A1
(43) Pub. Date: Aug. 22, 2019

(54) CANNABIS INFUSED GELATIN COMPOSITION AND METHOD

(71) Applicant: Shawn Garrels, Coquitlam (CA)

(72) Inventor: Shawn Garrels, Coquitlam (CA)

(21) Appl. No.: 15/899,265

(22) Filed: Feb. 19, 2018

(51) Int. Cl. A61K 36/185; A61K 47/42; A61K 47/36; A61K 47/10; A61K 9/00; A23L 29/281; A23L 33/105; A23L 2/02; A23L 2/56

(52) U.S. Cl. CPC A61K 36/185; A61K 47/42; A61K 47/36; A61K 47/10; A23V 2002/00; A23L 29/284; A23L 33/105; A23L 2/02; A23L 2/56; A61K 9/0056

(57) ABSTRACT

A cannabis infused gelatin composition includes at least one flavored gelatin powder, unflavored-gelatin-powder, cornstarch, glycerin, artificially fruit-flavored beverage, sweetener, and cannabis oil. The cannabis infused gelatin composition is useful for oral human consumption for imparting extracts found in cannabis plants to a user. In another embodiment, included herein is a berry flavored cannabis infused gelatin composition.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of gelatin compositions of existing art and more specifically relates to a cannabis infused gelatin composition.

### RELATED ART

A psychoactive drug or a chemical substance acts and affects brain function, resulting in alterations in perception, mood, consciousness, cognition, and behavior. Cannabis (containing THC chemical) is a psychoactive drug from a plant intended for medical or recreational use. These substances, when used, may purposefully alter one's consciousness, or as entheogens, for studying, spiritual, and/or shamanic purposes, etc.

Cannabis can be used and consumed in a variety of ways, such as edibles, smoking, and vaporizers. Studies have been conducted showing the negative effects smoking and vaporizers can have on the lungs. Such a method of administering the effects of cannabis may be undesirable and not needed in order to use the psychoactive drug. Furthermore, smoking and vaporizers may produce an objectionable smell. Therefore, a suitable solution is desired.

U.S. Pat. No. 9,833,408 to Allen Greenspoon relates to an orally administrable formulation. The described orally administrable formulation includes an orally administrable chewing gum formulation comprising a pharmaceutically acceptable gum base and particles of a pharmaceutical agent ranging in size from about 50 to about 2000 μm, wherein the formulation comprises about 0.5-30% by wt of the pharmaceutical agent particles. A liquid formulation comprising particles of a pharmaceutical agent is also provided.

### SUMMARY OF THE INVENTION

In view of the foregoing disadvantages inherent in the known gelatin composition art, the present disclosure provides a novel cannabis infused gelatin composition and method. The general purpose of the present disclosure, which will be described subsequently in greater detail, is to provide an effective cannabis infused gelatin composition and method.

A cannabis infused gelatin composition is disclosed herein. The cannabis infused gelatin composition includes the following: at least one flavored gelatin powder, unflavored-gelatin-powder, cornstarch, glycerin, artificially fruit-flavored beverage, sweetener, and cannabis oil. The cannabis infused gelatin composition may comprise in formulaic combination the at least one flavored gelatin powder, the unflavored-gelatin-powder, the cornstarch, the glycerin, the artificially fruit-flavored beverage, the sweetener, and the cannabis oil thereby creating an admixture. The cannabis infused gelatin composition may be suitably mixed for oral human consumption for imparting extracts found in cannabis plants to a user.

According to another embodiment, a method for producing a cannabis infused gelatin composition is also disclosed herein. The method for producing a cannabis infused gelatin composition includes a providing a cannabis oil comprising a sunflower lecithin and a cannabis-source; heating the cannabis oil to a minimum temperature of 190 degrees Fahrenheit and a maximum temperature of 210 degrees Fahrenheit while consistently stirring; mixing at least one flavored gelatin powder, unflavored-gelatin-powder, and cornstarch together; adding an artificially fruit-flavored beverage to the at least one flavored gelatin powder, the unflavored-gelatin-powder, and the cornstarch; adding a sweetener; heating the artificially fruit-flavored beverage, the at least one flavored gelatin powder, the unflavored-gelatin-powder, and the cornstarch; stirring the artificially fruit-flavored beverage, the at least one flavored gelatin powder, the unflavored-gelatin-powder, and the cornstarch until blended without granules; adding glycerin to the cannabis oil; blending the cannabis oil with the glycerin to the artificially fruit-flavored beverage, the at least one flavored gelatin powder, the unflavored-gelatin-powder, and the cornstarch thereby creating an admixture; and molding the admixture to a desired shape or size for oral human consumption.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a cannabis infused gelatin composition and method, constructed and operative according to the teachings of the present disclosure.

FIG. 1 is a perspective view of the cannabis infused gelatin composition during an ‘in-use’ condition, according to an embodiment of the disclosure.

FIG. 2 is a data table of the cannabis infused gelatin composition of FIG. 1, according to an embodiment of the present disclosure.

FIG. 3 is a data table of the cannabis infused gelatin composition of FIG. 1, according to an embodiment of the present disclosure.

FIG. 4 is a perspective view of the cannabis infused gelatin composition of FIG. 1, according to an embodiment of the present disclosure.

FIG. 5 is a flow diagram illustrating a method for producing a cannabis infused gelatin composition, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a gelatin composition and more particularly to a cannabis infused gelatin composition and method as used to improve the oral administration of cannabis infused edibles.

Generally, the cannabis infused gelatin composition and method may be utilized to produce 5 ml, 10 ml, 35 ml, 50 ml, 75 ml, 100 ml, and 300 ml of THC found in cannabis oil. The amount of artificially fruit-flavored beverage, at least one flavored juice, and at least one gelatin powder used may vary based on the amount of cannabis oil utilized. Further, it should be noted that the at least one flavored gelatin powder may be similar to commercially sold JELL-O®, and the artificially fruit-flavored beverage may be similar to FRUITOPIA®. The cannabis infused gelatin composition may be strawberry flavored or the like.

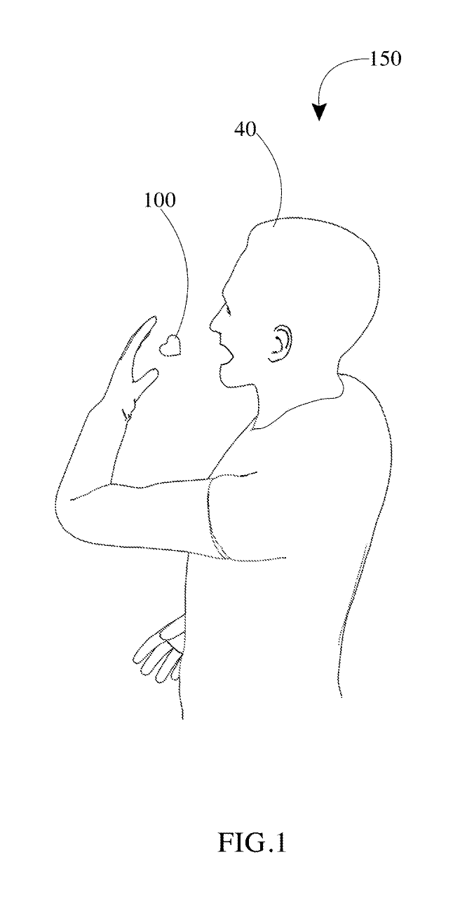

Referring now more specifically to the drawings by numerals of reference, there is shown in FIGS. 1-4, various views of a cannabis infused gelatin composition 100.

FIG. 1 shows a cannabis infused gelatin composition 100 during an ‘in-use’ condition 50, according to an embodiment of the present disclosure. Here, the cannabis infused gelatin composition 100 may be beneficial for oral consumption by a user 40 to administer the effects of cannabis. As illustrated, the cannabis infused gelatin composition 100 may include at least one flavored gelatin powder 110, unflavored-gelatin-powder 112, cornstarch 114, glycerin 116, artificially fruit-flavored beverage 118, sweetener 122, and cannabis oil 120. The cannabis oil 120 may comprise at least one oil (i.e., sunflower lecithin) and a cannabis-source (i.e., shatter, oil, dissalite, etc.). In one embodiment, the cannabis infused gelatin composition 100 may be included in a strawberry flavor with the formulaic combination provided.

The cannabis infused gelatin composition 100 may comprise in formulaic combination the at least one flavored gelatin powder 110, the unflavored-gelatin-powder 112, the cornstarch 114, the glycerin 116, the artificially fruit-flavored beverage 118, the sweetener 122, and the cannabis oil 120 thereby creating an admixture. The cannabis infused gelatin composition 100 may be suitably mixed for oral human consumption for imparting extracts found in cannabis plants to the user 40.

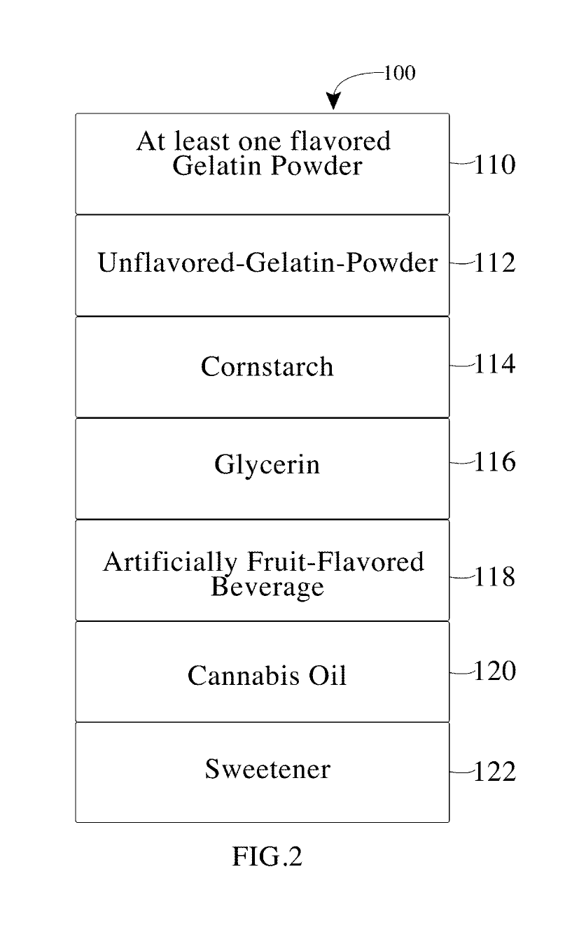

FIG. 2 shows the cannabis infused gelatin composition 100 of FIG. 1, according to an embodiment of the present disclosure. As above, the cannabis infused gelatin composition 100 may include the at least one flavored gelatin powder 110, the unflavored-gelatin-powder 112, the cornstarch 114, the glycerin 116, the artificially fruit-flavored beverage 118, the sweetener 122, and the cannabis oil 120. The sweetener 122 may include agave syrup or any other suitable sweetener.

In accordance to the present embodiment, the at least one flavored gelatin powder 110 may include a strawberry-flavor-gelatin. The at least one flavored gelatin powder 110 of the strawberry-flavor-gelatin may comprise a minimum of 510 grams and a maximum of 1, 870 grams of the admixture. In addition to the strawberry-flavor-gelatin, the at least one flavored gelatin powder 110 may include a raspberry-flavor-gelatin. The at least one flavored gelatin powder 110 of the raspberry-flavor-gelatin may comprise a minimum of 170 grams and a maximum of 255 grams of the admixture.

The unflavored-gelatin-powder 112 may comprise a minimum of 200 mg and a maximum of 700 mg of the admixture. The cornstarch 114 may comprise a minimum of 3 teaspoons and a maximum of 9 teaspoons of the admixture. The glycerin 116 may comprise 180 ml of the admixture. Further, the artificially fruit-flavored beverage 118 may comprise a minimum of 1,420 ml and a maximum of 2,840 ml of the admixture. The sweetener 122 may comprises a minimum of 130 ml and a maximum of 260 ml of the admixture

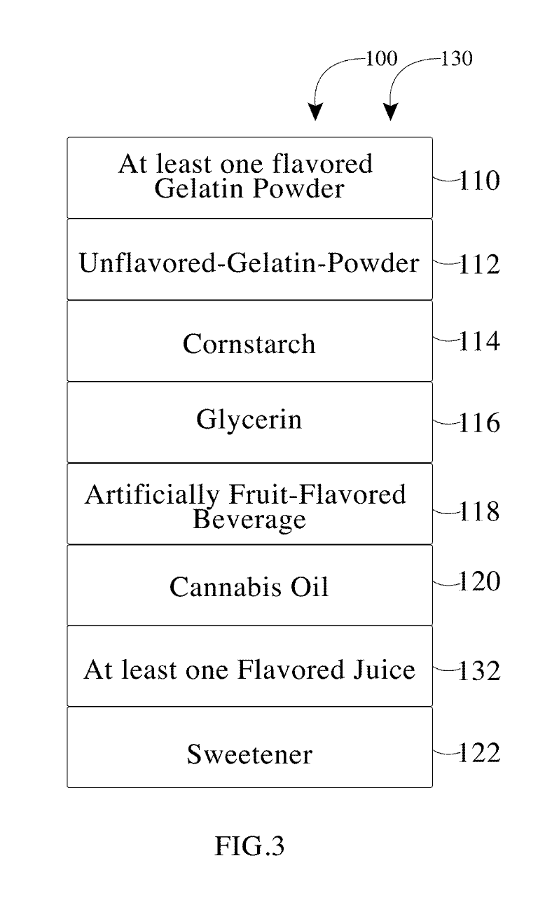

Referring now to FIG. 3, a data table of the cannabis infused gelatin composition 100 of FIG. 1, according to another embodiment of the present disclosure. The cannabis infused gelatin composition 100 may include additional liquid for creating a berry-flavored cannabis infused gelatin composition 130. The liquid may include at least one flavored juice 132. The berry flavored cannabis infused gelatin composition 130 may have, in formulaic combination, the at least one flavored gelatin powder 110, the unflavored-gelatin-powder 112, the cornstarch 114, the glycerin 116, the artificially fruit-flavored beverage 118, the sweetener 122, the cannabis oil 120, and the at least one flavored juice 132 thereby creating the admixture. The berry-flavored cannabis infused gelatin composition 130 may be suitably mixed to provide for the oral human consumption for imparting the extracts found in the cannabis plants to the user 40.

In accordance to the present embodiment, the at least one flavored juice 132 may be a grape-flavor-juice. The at least one flavored juice 132 being the grape-flavor-juice may comprise at least 1,065 ml of the admixture. The at least one flavored juice 132 may further include a berry-flavor-juice. The at least one flavored juice 132 being the berry-flavor-juice may comprise at least 1,065 ml of the admixture.

The at least one flavored gelatin powder 110 for the berry flavored cannabis infused gelatin composition 130 may include the strawberry-flavor-gelatin, the raspberry-flavor-gelatin, and a grape-flavor-gelatin. The at least one flavored gelatin powder 110 of the grape-flavor-gelatin may comprise at least 255 grams of the admixture. Other flavors may be used.

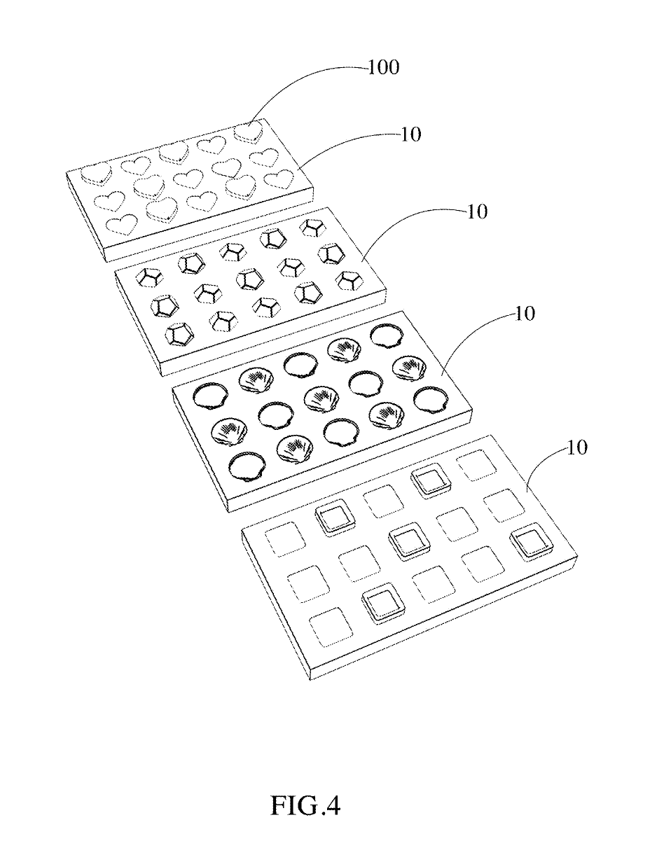

FIG. 4 is a perspective view of the cannabis infused gelatin composition of FIG. 1, according to an embodiment of the present disclosure. The cannabis infused gelatin composition 100 may be applied to a mold 10 enabling the admixture to be a desired shape or size for oral human consumption.

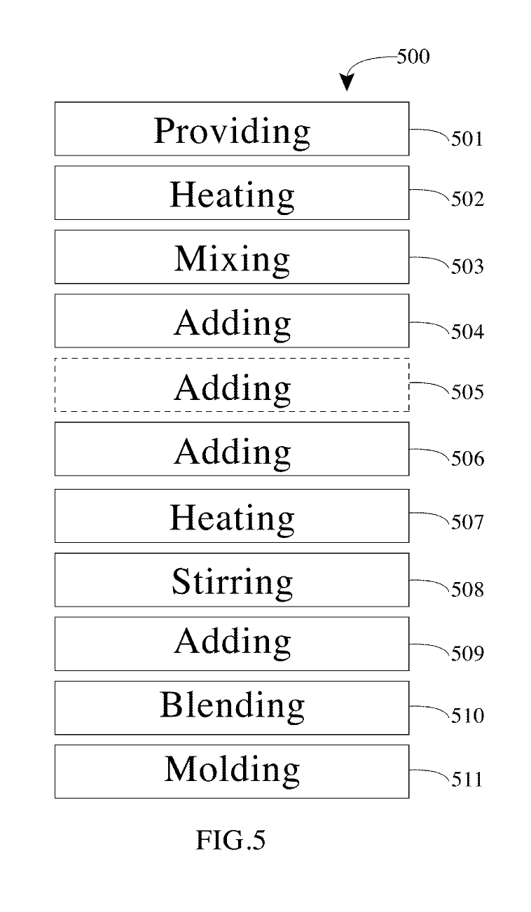

Referring now to FIG. 5 showing a flow diagram illustrating a method for producing a cannabis infused gelatin composition 500, according to an embodiment of the present disclosure. In particular, the method for producing a cannabis infused gelatin composition 500 may include one or more components or features of the cannabis infused gelatin composition 100 as described above. As illustrated, the method for producing a cannabis infused gelatin composition 500 may include the steps of: step one 501, providing a cannabis oil comprising a sunflower lecithin and a cannabis-source; step two 502, heating the cannabis oil to a minimum temperature of 190 degrees Fahrenheit and a maximum temperature of 210 degrees Fahrenheit while consistently stirring; step three 503, mixing at least one flavored gelatin powder, unflavored-gelatin-powder, and cornstarch together; step four 504, adding an artificially fruit-flavored beverage to the at least one flavored gelatin powder, the unflavored-gelatin-powder, and the cornstarch; step five 505, adding at least one flavored juice with the artificially fruit-flavored beverage thereby producing a berry flavored cannabis infused gelatin composition; step six 506, adding a sweetener; step seven 507, heating the artificially fruit-flavored beverage, the at least one flavored gelatin powder, the unflavored-gelatin-powder, and the cornstarch; step eight 508, stirring the artificially fruit-flavored beverage, the at least one flavored gelatin powder, the unflavored-gelatin-powder, and the cornstarch until blended without granules; step nine 509, adding glycerin to the cannabis oil; step ten 510, blending the cannabis oil with the glycerin to the artificially fruit-flavored beverage, the at least one flavored gelatin powder, the unflavored-gelatin-powder, and the cornstarch thereby creating an admixture; and step eleven 511, molding the admixture to a desired shape or size for oral human consumption.

It should be noted that step five 505 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for producing a cannabis infused gelatin composition 500 (e.g., different step orders within above-mentioned list, elimination or addition of certain steps, including or excluding certain maintenance steps, etc.) are taught herein.

Those with ordinary skill in the art will now appreciate that upon reading this specification and by their understanding the art of gelatin compositions as described herein, methods of producing a cannabis infused gelatin composition 500 will be understood by those knowledgeable in such art.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A cannabis infused gelatin composition comprising:
at least one flavored gelatin powder;
unflavored-gelatin-powder;
cornstarch;
glycerin;
artificially fruit-flavored beverage;
sweetener; and
cannabis oil;
wherein said cannabis infused gelatin composition comprises in formulaic combination said at least one flavored gelatin powder, said unflavored-gelatin-powder, said cornstarch, said glycerin, said artificially fruit-flavored beverage, said sweetener, and said cannabis oil thereby creating an admixture; and
wherein said cannabis infused gelatin composition is suitably mixed for oral human consumption for imparting extracts found in cannabis plants to a user.

2. The cannabis infused gelatin composition of claim 1, wherein said at least one flavored gelatin powder includes a strawberry-flavor-gelatin.

3. The cannabis infused gelatin composition of claim 2, wherein said at least one flavored gelatin powder of said strawberry-flavor-gelatin comprises a minimum of 510 grams and a maximum of 1,870 grams of said admixture.

4. The cannabis infused gelatin composition of claim 1, wherein said at least one flavored gelatin powder includes a raspberry-flavor-gelatin.

5. The cannabis infused gelatin composition of claim 4, wherein said at least one flavored gelatin powder of said raspberry-flavor-gelatin comprises a minimum of 170 grams and a maximum of 255 grams of said admixture.

6. The cannabis infused gelatin composition of claim 1, wherein said unflavored-gelatin-powder comprises a minimum of 200 mg and a maximum of 700 mg of said admixture.

7. The cannabis infused gelatin composition of claim 1, wherein said cornstarch comprises a minimum of 3 teaspoons and a maximum of 9 teaspoons of said admixture.

8. The cannabis infused gelatin composition of claim 1, wherein said glycerin comprises 180 ml of said admixture.

9. The cannabis infused gelatin composition of claim 1, wherein said artificially fruit-flavored beverage comprises a minimum of 1,420 ml and a maximum of 2,840 ml of said admixture.

10. The cannabis infused gelatin composition of claim 1, wherein said admixture further includes a berry flavored cannabis infused gelatin composition having in formulaic combination said at least one flavored gelatin powder, said unflavored-gelatin-powder, said cornstarch, said glycerin, said artificially fruit-flavored beverage, said cannabis oil, and at least one flavored juice thereby creating an admixture; and
wherein said berry flavored cannabis infused gelatin composition is suitably mixed to provide for said oral human consumption for imparting said extracts found in said cannabis plants to said user.

11. The cannabis infused gelatin composition of claim 10, wherein said at least one flavored juice is a grape-flavor-juice.

12. The cannabis infused gelatin composition of claim 11, wherein said at least one flavored juice being said grape-flavor-juice comprises at least 1,065 ml of said admixture.

13. The cannabis infused gelatin composition of claim 10, wherein said at least one flavored juice is a berry-flavor-juice.

14. The cannabis infused gelatin composition of claim 13, wherein said at least one flavored juice being said berry-flavor-juice comprises at least 1,065 ml of said admixture.

15. The cannabis infused gelatin composition of claim 10, wherein said at least one flavored gelatin powder for said berry flavored cannabis infused gelatin composition includes said strawberry-flavor-gelatin, said raspberry-flavor-gelatin, and a grape-flavor-gelatin.

16. The cannabis infused gelatin composition of claim 15, wherein said at least one flavored gelatin powder of said grape-flavor-gelatin comprises at least 255 grams of said admixture.

17. A cannabis infused gelatin composition comprising:
at least one flavored gelatin powder;
unflavored-gelatin-powder;
cornstarch;
glycerin;
artificially fruit-flavored beverage;
sweetener; and
cannabis oil;
wherein said cannabis infused gelatin composition comprises in formulaic combination said at least one flavored gelatin powder, said unflavored-gelatin-powder, said cornstarch, said glycerin, said artificially fruit-flavored beverage, said sweetener, and said cannabis oil thereby creating an admixture;
wherein said cannabis infused gelatin composition is suitably mixed for oral human consumption for imparting extracts found in cannabis plants to a user;
wherein said sweetener comprises a minimum of 130 ml and a maximum of 260 ml of said admixture;
wherein said at least one flavored gelatin powder includes a strawberry-flavor-gelatin;
wherein said at least one flavored gelatin powder of said strawberry-flavor-gelatin comprises a minimum of 510 grams and a maximum of 1,870 grams of said admixture;
wherein said at least one flavored gelatin powder includes a raspberry-flavor-gelatin;
wherein said at least one flavored gelatin powder of said raspberry-flavor-gelatin comprises a minimum of 170 grams and a maximum of 255 grams of said admixture;
wherein said unflavored gelatin powder comprises a minimum of 200 mg and a maximum of 700 mg of said admixture;
wherein said cornstarch comprises a minimum of 3 teaspoons and a maximum of 9 teaspoons of said admixture;
wherein said glycerin comprises 180 ml of said admixture;
wherein said artificially fruit-flavored beverage comprises a minimum of 1,420 ml and a maximum of 2,840 ml of said admixture; and
wherein said cannabis infused gelatin composition includes additional liquid for creating a berry flavored cannabis infused gelatin composition.

18. The cannabis infused gelatin composition of claim 17, wherein said liquid includes at least one flavored juice.

19. A method for producing a cannabis infused gelatin composition, the method comprising the steps of:
providing a cannabis oil comprising a sunflower lecithin and a cannabis-source,
heating said cannabis oil to a minimum temperature of 190 degrees Fahrenheit and a maximum temperature of 210 degrees Fahrenheit while consistently stirring,
mixing at least one flavored gelatin powder, unflavored-gelatin-powder, and cornstarch together,
adding an artificially fruit-flavored beverage to said at least one flavored gelatin powder, said unflavored-gelatin-powder, and said cornstarch,
adding a sweetener,
heating said artificially fruit-flavored beverage, said at least one flavored gelatin powder, said unflavored-gelatin-powder, and said cornstarch,
stirring said artificially fruit-flavored beverage, said at least one flavored gelatin powder, said unflavored-gelatin-powder, and said cornstarch until blended without granules,
adding glycerin to said cannabis oil,
blending said cannabis oil with said glycerin to said artificially fruit-flavored beverage, said at least one flavored gelatin powder, said unflavored-gelatin-powder, and said cornstarch thereby creating an admixture, and
molding said admixture to a desired shape or size for oral human consumption.

20. The method of claim 19, further comprising the steps of
adding at least one flavored juice with said artificially fruit-flavored beverage thereby producing a berry flavored cannabis infused gelatin composition.
